(19) United States
(12) Patent Application Publication
(10) Pub. No.: US 2022/0102008 A1
(43) Pub. Date: Mar. 31, 2022

(54) METHODS AND SYSTEMS FOR PLACEBO RESPONSE MODELING

(71) Applicant: Genentech, Inc., South San Francisco, CA (US)

(72) Inventors: Tong LU, South San Francisco, CA (US); Angelica Linnea QUARTINO, South San Francisco, CA (US); Meina Tao TANG, South San Francisco, CA (US); Wenhui ZHANG, South San Francisco, CA (US); Rui ZHU, South San Francisco, CA (US); Matts Lennart KAAGEDAL, South San Francisco, CA (US); Sonoko KAWAKATSU, South San Francisco, CA (US)

(21) Appl. No.: 17/482,342

(22) Filed: Sep. 22, 2021

Related U.S. Application Data
(60) Provisional application No. 63/085,807, filed on Sep. 30, 2020.
(60) Provisional application No. 63/163,316, filed on Mar. 19, 2021.

(51) Int. Cl. G16H 50/30; G16H 50/20; G06F 17/18

(52) U.S. Cl. CPC G16H 50/30; G06F 17/18; G16H 50/20

(57) ABSTRACT

Embodiments described herein provide methods and systems for placebo response modeling. The methods and systems generally operate by using one or more statistical operations (such as one or more pharmacometric operations or artificial intelligence (AI) operations) to predict sets of predicted scores that are relevant to ulcerative colitis (UC) and that correspond to a set of time points.

### CROSS-REFERENCE

The present application claims priority to U.S. Provisional Application No. 63/085,807, entitled “METHODS AND SYSTEMS FOR PLACEBO RESPONSE MODELING,” filed on Sep. 30, 2020, and U.S. Provisional Application No. 63/163,316, entitled “METHODS AND SYSTEMS FOR PLACEBO RESPONSE MODELING,” filed on Mar. 19, 2021, which applications are entirely incorporated herein by reference for all purposes.

### TECHNICAL FIELD

The present disclosure relates generally to methods and systems for placebo response modeling.

### BACKGROUND

Clinical trials for ulcerative colitis (UC) have relatively high placebo response rates in comparison to clinical trials for other diseases. Thus, there is a need for methods and systems for placebo response modeling in UC studies, in order to better distinguish drug response from placebo response.

### BRIEF DESCRIPTION OF THE DRAWINGS

FIG. 1A is a simplified diagram of a process flow for placebo response modeling in ulcerative colitis (UC) patient populations, in accordance with various embodiments.

FIG. 1B is a simplified diagram of a method for placebo response modeling in UC patient populations, in accordance with various embodiments.

FIG. 2 is a block diagram of a system for placebo response modeling, in accordance with various embodiments.

FIG. 3 is a block diagram of a computer system, in accordance with various embodiments.

FIG. 4 shows a schematic of an exemplary UC model structure, in accordance with various embodiments.

FIG. 5A shows categorical VPCs for post-baseline RB+SF subscores, in accordance with various embodiments.

FIG. 5B shows categorical VPCs for post-baseline ENDO subscores, in accordance with various embodiments.

FIG. 5C shows categorical VPCs for post-baseline PGA subscores, in accordance with various embodiments.

FIG. 5D shows categorical VPCs for post-baseline end of induction and maintenance phase dropout, in accordance with various embodiments.

FIG. 6 shows continuous VPC of modified MCS over time in patients remaining in the trial, in accordance with various embodiments.

FIG. 7 shows a comparison of model predicted modified MCS with dropout and without dropout, in accordance with various embodiments.

In various embodiments, not all of the depicted components in each figure may be required, and various embodiments may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

Ulcerative colitis (UC) is a type of inflammatory bowel disease characterized by chronic inflammation of the large intestine. The clinical course is unpredictable and is characterized by periods of remission and relapse. Currently, there is no cure for UC, and treatment is focused on minimizing symptoms and disease progression. Clinical trials for the development of new treatments in this population may face the challenge of high placebo response rates, which may make it difficult to draw conclusions about treatment-related response. In addition, evaluating efficacy of a new agent in pediatric trials may raise severe ethical and feasibility issues if including a placebo/standard of care (SoC) arm. When there is already efficacy data for an active treatment in adults, the use of a placebo/SoC arm in pediatric trials may be avoided because it may expose patients to a known inferior treatment. Enrollment in a pediatric trial may also be difficult due to the lower prevalence of UC in children. A model that describes longitudinal placebo response may help to guide the design of clinical trials (e.g. sample size calculations), and may provide information about expected placebo response in trials where a placebo/SoC arm is not available. In addition, such a model may allow for the evaluation of intrinsic and extrinsic factors that can influence placebo response in patients with UC.

To address the need for methods and systems for placebo response modeling in UC studies, this specification describes exemplary methods and systems for longitudinal placebo response modeling, such as in UC patient populations. The methods and systems generally operate by using one or more statistical models (such as one or more pharmacometric models or artificial intelligence (AI) models) to generate sets of predicted scores that are relevant to UC and that correspond to a set of time points.

In particular, a Mayo clinical score (MCS) scoring system is widely used in UC clinical trials. The MCS is a discrete number comprised of four subscores: rectal bleeding (RB), stool frequency (SF), physician's global assessment (PGA), and endoscopy (ENDO) subscores. Each subscore may be scored from 0 to 3, and the subscores may be added to yield the MCS, which may range from 0 to 12, with higher scores indicating increased disease severity. Excluding the ENDO subscore from the MCS gives the partial MCS, and excluding the PGA subscore gives the modified MCS. Existing placebo response prediction models may only predict the entire MCS, merely outputting score predictions ranging from 0 to 12. In other words, current placebo response prediction models may not account for the individual subscores and nuances in predicting those subscores and may not predict the partial MCS or modified MCS. Consequently, current placebo response models also may not reflect relationships, linkages, or covariance between the subscores, or the impact of one or more subscores on another subscore. In short, existing placebo response prediction models may merely output scores ranging from 0 to 12, without insights into the individual subscores that may have contributed to the resultant 0 to 12 MCS score.

Furthermore, existing placebo response prediction models may generate score predictions from medical examination and patient-reported outcomes in relation to past MCS scores. Such medical examination and patient-reported outcomes may be reported at discrete time points. As a result, existing placebo response prediction models may not provide continuous predictive scores or may not show UC disease progression.

Modeling the time course of the modified MCS may face the additional challenge of complex trial design. For studies with both an induction and maintenance phase, patients in the placebo/SoC arm may be removed from the trial at the end of induction or may progress to the maintenance phase through a variety of mechanisms. Patients that progress into the maintenance phase may remain in the placebo/SoC arm, be re-randomized into a placebo/SoC or treatment arm, or be placed into a treatment arm based on responder status. Models that account for this potential removal of patients from the placebo/SoC arm at the end of induction (such as the methods and systems described herein) may allow for a more accurate characterization of longitudinal placebo response through the maintenance phase. Because an investigational therapy must demonstrate efficacy in both the induction and maintenance phase, a better understanding of placebo response in the maintenance phase is needed to aid the evaluation of drug effect.

The present embodiments provide a longitudinal UC placebo response prediction model that accounts for relationships, linkages, or covariance between the MCS subscores, as well as disease progression. In doing so, the present embodiments may provide more accurate placebo response prediction scores, as well as insights as to how individual subscores behave over time. Further, the present embodiments may allow discernment of how individual subscores behave in relation to one another, and may provide the basis for further predictive capabilities.

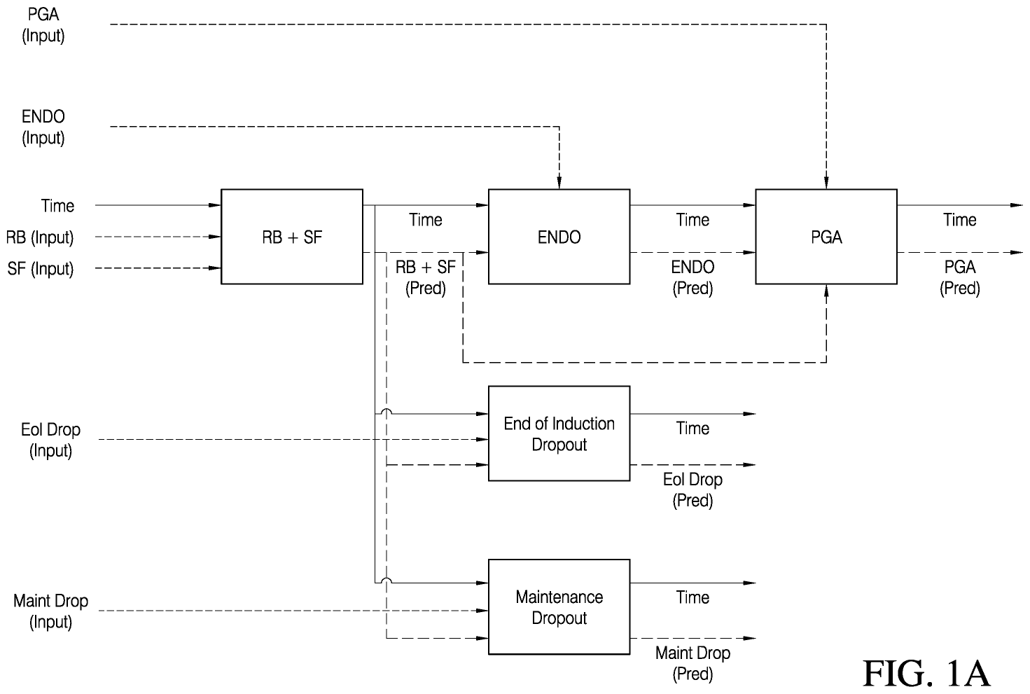

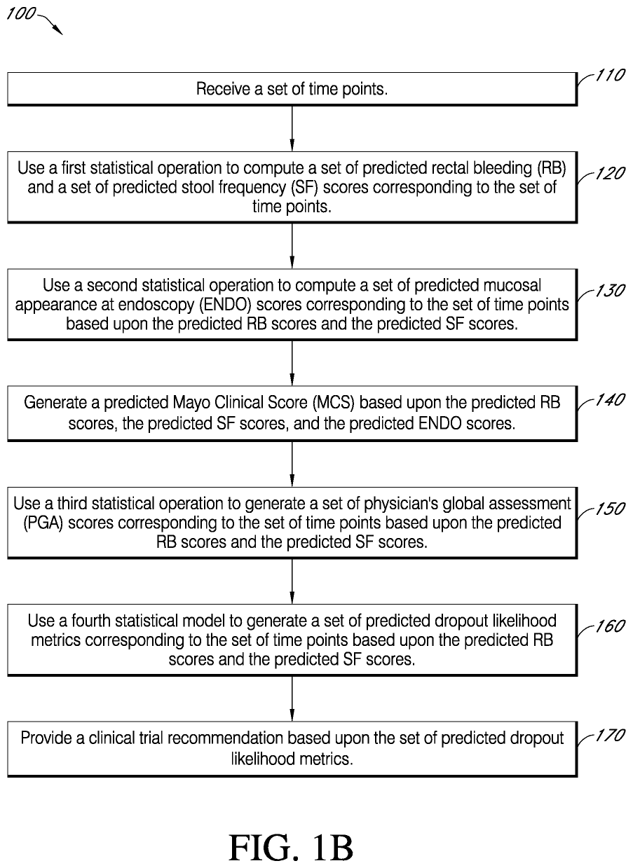

FIG. 1A is a simplified exemplary diagram of a process flow for placebo response modeling in UC patient populations. According to various embodiments, one or more processors receive a set of time points. The one or more processors may perform at least one first statistical operation to predict a set of predicted RB scores and SF scores corresponding to the set of time points. The predicted RB and SF scores may correspond to RB and SF scores in the MCS scoring system. For instance, a predicted RB score of 0 may indicate a prediction of no rectal bleeding, a predicted RB score of 1 may indicate a prediction of visible blood in stool less than half the time, a predicted RB score of 2 may indicate a prediction of visible blood in stool more than half the time, and a predicted RB score of 3 may indicate a prediction of passing blood alone. A predicted SF score of 0 may indicate a prediction of normal stool frequency, a predicted SF score of 1 may indicate a prediction of 1-2 stools per day more than normal, a predicted SF score of 2 may indicate a prediction of 3-4 stools per day more than normal, and a predicted SF score of 3 may indicate a prediction of more than 4 stools per day more than normal. In various embodiments, the first statistical operation may predict a set of predicted distributions of RB scores and a set of predicted distributions of SF scores. In some cases, the RB scores and the SF scores may be summed to yield a predicted set of RB+SF scores.

The one or more processors may perform at least one second statistical operation to use the predicted RB scores and the predicted SF scores to predict a set of predicted ENDO scores. The predicted ENDO scores may correspond to ENDO scores in the MCS scoring system. For instance, a predicted ENDO score of 0 may indicate a prediction of normal or inactive UC, a predicted ENDO score of 1 may indicate a prediction of mild UC (shown, for instance, by erythema, decreased vascular pattern, and/or mild friability), a predicted ENDO score of 2 may indicate a prediction of moderate UC (shown, for instance, by marked erythema, absent vascular pattern, friability, and/or erosions), and a predicted ENDO score of 3 may indicate a prediction of severe UC (shown, for instance, by spontaneous bleeding and/or ulceration). In various embodiments, the second statistical operation may predict a set of predicted distributions of ENDO scores.

The predicted ENDO scores may allow for more frequent estimation of ENDO scores in a subject population during a clinical trial. It may be difficult to perform frequent endoscopic assessments on subjects during a clinical trial, as endoscopic procedures may be invasive and painful. As such, the number of ENDO data points that may actually be collected may be smaller than desired. However, RB and SF measurements may be collected far more frequently. As such, the predicted ENDO scores may have much greater time resolution than might be possible by taking actual endoscopic measurements.

The one or more processors may perform at least one third statistical operation to use the predicted RB scores, the predicted SF scores, and the predicted ENDO scores to predict a set of predicted PGA scores. The predicted PGA scores may correspond to PGA scores in the MCS scoring system. For instance, a predicted PGA score of 0 may indicate a prediction of normal, a predicted PGA score of 1 may indicate a prediction of mild UC a predicted PGA score of 2 may indicate a prediction of moderate UC, and a predicted PGA score of 3 may indicate a prediction of severe UC. In various embodiments, the third statistical operation may predict a set of predicted distributions of PGA scores.

The one or more processors may perform at least one fourth statistical operation to use the predicted RB scores and the predicted SF scores to predict a set of dropout likelihoods in a clinical trial. The dropout likelihoods may be used to inform clinical design by allowing estimates, for instance, of how large a cohort may be required for statistical discrimination in the clinical trial.

Any 1, 2, 3, or 4 of the first, second, third, and fourth statistical operations may comprise an artificial intelligence (AI) model or a pharmacometric model. For instance, any of the first, second, third, and fourth statistical operations may comprise a proportional odds (PO) modeling operation or a logistic regression operation. For example, any of the first, second, third, and fourth statistical operations may be performed using the PO modeling operations described in Equations (1) and (2) herein. The first, second, third, and fourth statistical operations may be trained using data, such as MCS scores or modified MCS scores from prior UC clinical trials.

FIG. 1B is a simplified diagram of a method 100 for placebo response modeling in UC patient populations. The method 100 may comprise operations similar to the computer-implemented operations described herein with respect to FIG. 1A. The method 100 may comprise a first step 110 of receiving a set of time points, as described herein with respect to FIG. 1A.

The method 100 may comprise a second step 120 of using a first statistical operation to compute a set of predicted RB scores and a set of predicted SF scores corresponding to the set of time points, as described herein with respect to FIG. 1A.

The method 100 may comprise a third step 130 of using a second statistical operation to compute, using the predicted RB scores and the predicted SF scores, a set of predicted ENDO scores corresponding to the set of time points based upon the predicted RB scores and the predicted SF scores, as described herein with respect to FIG. 1A.

The method 100 may comprise a fourth step 140 of generating a predicted MCS based upon the predicted RB scores, the predicted SF scores, and the predicted ENDO scores, as described herein with respect to FIG. 1A.

The method 100 may comprise a fifth step 150 of using a third statistical operation to generate a set of predicted PGA scores corresponding to the set of time points based upon the predicted RB scores, the predicted SF scores, and the predicted ENDO scores, as described herein with respect to FIG. 1A.

The method 100 may comprise a sixth step 160 of using a fourth statistical model to generate a set of predicted dropout likelihood metrics corresponding to the set of time points based upon the predicted RB scores and the predicted SF scores, as described herein with respect to FIG. 1A.

The method 100 may comprise a seventh step 170 of providing a clinical trial recommendation based upon the set of predicted dropout likelihood metrics, as described herein with respect to FIG. 1A.

### Computer-Implemented System

In various embodiments, at least a portion of the methods for placebo response monitoring can be implemented via software, hardware, firmware, or a combination thereof.

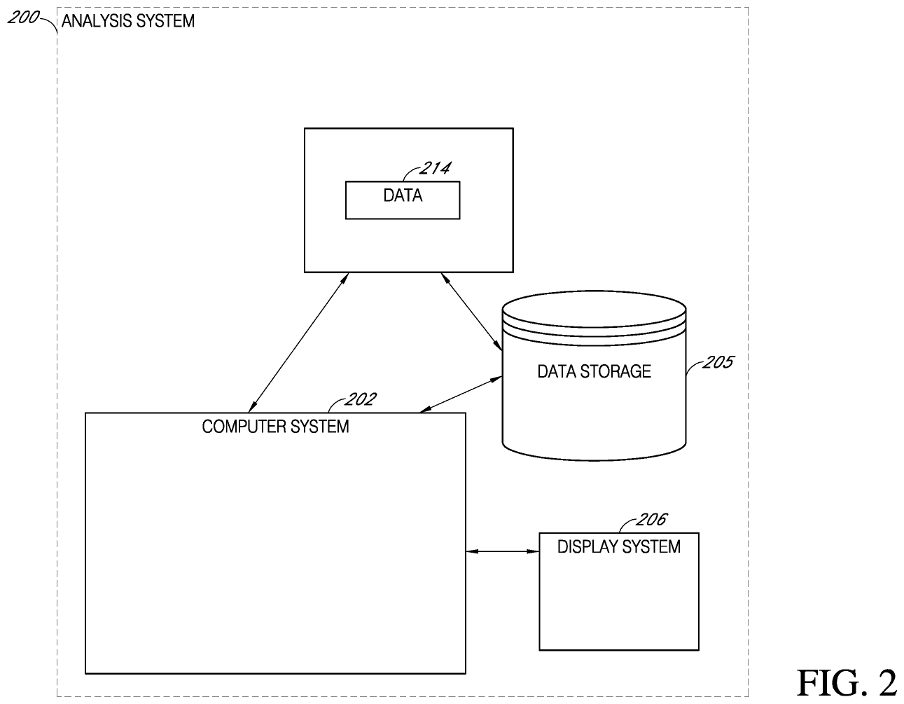

That is, as depicted in FIG. 2, the methods disclosed herein can be implemented on a system 200 for placebo response modeling. The system 200 may comprise a computer system such as computer system 202 (e.g., a computing device/analytics server). In various embodiments, the computer system 202 can be communicatively connected to a data storage 205 and a display system 206 via a direct connection or through a network connection (e.g., LAN, WAN, Internet, etc.). The computer system 202 can be configured to receive data, such as MCS data or modified MCS data described herein. It should be appreciated that the computer system 202 depicted in FIG. 2 can comprise additional engines or components as needed by the particular application or system architecture.

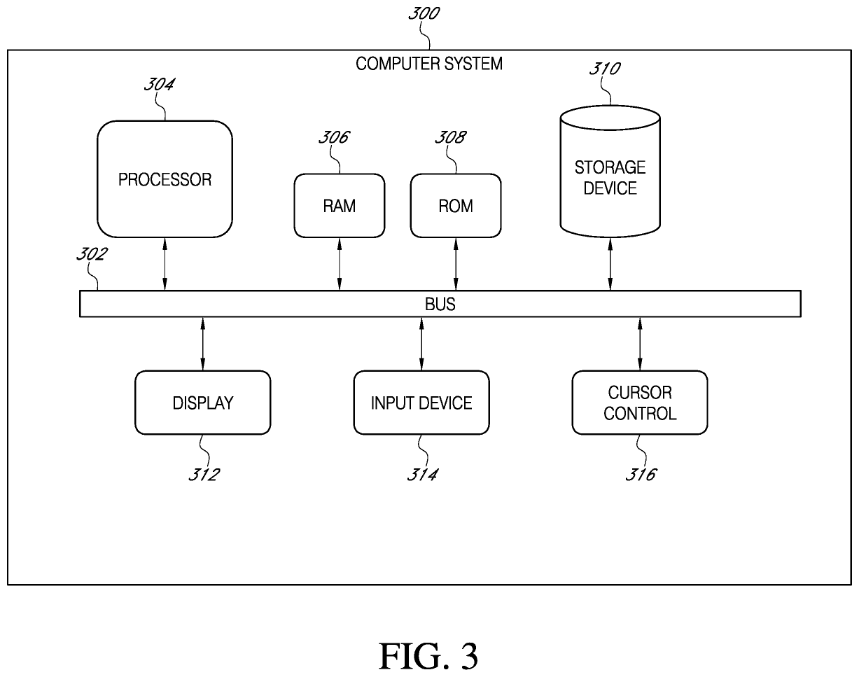

FIG. 3 is a block diagram of a computer system in accordance with various embodiments. Computer system 300 may be an example of one implementation for computer system 202 described above in FIG. 2. In one or more examples, computer system 300 can include a bus 302 or other communication mechanism for communicating information, and a processor 304 coupled with bus 302 for processing information. In various embodiments, computer system 300 can also include a memory, which can be a random access memory (RAM) 306 or other dynamic storage device, coupled to bus 302 for determining instructions to be executed by processor 304. Memory also can be used for storing temporary variables or other intermediate information during execution of instructions to be executed by processor 304. In various embodiments, computer system 300 can further include a read only memory (ROM) 308 or other static storage device coupled to bus 302 for storing static information and instructions for processor 304. A storage device 310, such as a magnetic disk or optical disk, can be provided and coupled to bus 302 for storing information and instructions.

In various embodiments, computer system 300 can be coupled via bus 302 to a display 312, such as a cathode ray tube (CRT) or liquid crystal display (LCD), for displaying information to a computer user. An input device 314, including alphanumeric and other keys, can be coupled to bus 302 for communicating information and command selections to processor 304. Another type of user input device is a cursor control 316, such as a mouse, a joystick, a trackball, a gesture input device, a gaze-based input device, or cursor direction keys for communicating direction information and command selections to processor 304 and for controlling cursor movement on display 312. This input device 314 typically has two degrees of freedom in two axes, a first axis (e.g., x) and a second axis (e.g., y), that allows the device to specify positions in a plane. However, it should be understood that input devices 312 allowing for three-dimensional (e.g., x, y and z) cursor movement are also contemplated herein.

Consistent with certain implementations of the present teachings, results can be provided by computer system 300 in response to processor 304 executing one or more sequences of one or more instructions contained in RAM 306. Such instructions can be read into RAM 306 from another computer-readable medium or computer-readable storage medium, such as storage device 310. Execution of the sequences of instructions contained in RAM 306 can cause processor 304 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 304 for execution. Such a medium can take many forms, including but not limited to, non-volatile media, volatile media, and transmission media. Examples of non-volatile media can include, but are not limited to, optical, solid state, magnetic disks, such as storage device 310. Examples of volatile media can include, but are not limited to, dynamic memory, such as RAM 306. Examples of transmission media can include, but are not limited to, coaxial cables, copper wire, and fiber optics, including the wires that comprise bus 302.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 304 of computer system 300 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 300 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 300, whereby processor 304 would execute the analyses and determinations provided by these engines, subject to instructions provided by any one of, or a combination of, the memory components RAM 306, ROM, 308, or storage device 310 and user input provided via input device 314.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

Although specific embodiments and applications of the disclosure have been described in this specification, these embodiments and applications are exemplary only, and many variations are possible.

### EXAMPLES

### Example 1: UC Longitudinal Model

A longitudinal model that describes the subscores of the MCS as separate endpoints during both induction and maintenance phases of a clinical trial in UC was developed. The model was also designed to improve estimates in the maintenance phase by accounting for the complex clinical trial design in UC, where patients may be removed from the placebo arm at the end of the induction phase. The effect of intrinsic and extrinsic factors on the MCS subscores were also evaluated.

All available longitudinal, patient-level placebo/SoC arm data from clinical trials for UC were extracted from the TransCelerate database. This database is readily accessible by member companies and contains de-identified, patient-level placebo/SoC arm clinical trial data for various non-oncology indications including UC. Data were pooled from five randomized, double-blind, placebo-controlled, multicenter phase 2 and 3 trials for moderate to severe active UC. The modeling dataset was assembled and visualized using the statistical software R (version 3.6.0). Separate subscore and dropout models were developed. The linkages between these separate model components were subsequently explored to develop a complete model that can estimate the modified MCS or MCS over time. The subscores of the MCS were modeled using separate PO models. Each PO model estimates the cumulative probability of having an observation Y, that is greater than or equal to a given score m. Logit transformations were used to ensure probabilities fell within the range from 0 to 1. The general PO model structure can be represented by Equation (1):

Logit[P(Y≥m|ηᵢ]=αₘ+PLB+ηᵢ (1)

Here, αₘ is the intercept, PLB is the placebo effect, and ηᵢ is the interindividual variability (IIV). In the model, P(Y≥0)=1 and αₘ for m>1 was reparametrized using a value DFₘ<0 to ensure that P(Y≥m)>P(Y≥m+1). DFₘ was the difference between αₘ and αₘ₊₁ and is given by Equation (2):

αₘ=αₘ₋₁+DFM,m>0 (2)

Baseline values for the subscores were included as covariates on the intercept parameter of the respective subscore model. Additionally potential effects of baseline covariates: prior exposure to tumor necrosis factor-α (TNF-α) antagonists, age, c-reactive protein (CRP), albumin, smoking status, concomitant medications, and steroid use were evaluated on the intercept parameter using a stepwise covariate modeling (SCM) approach. Covariates that resulted in a reduction in the objective function value (OFV) of greater than 3.84 (p<0.05 for one additional degree of freedom) were retained in the forward selection, and those that resulted in a change in OFV of greater than 10.8 (p<0.001) were retained in the backward elimination. Because individual-level information on concomitant medications and steroid use were not readily available for model building, summary-level information for each study on concomitant medications required before baseline and permitted during the study, and the proportion of placebo/SoC arm patients using steroids at baseline were used for covariate modeling.

For the trials that included both an induction and a maintenance phase, a dropout model was implemented to reflect the progression of only a portion of the placebo/SoC arm patients from the induction phase into the maintenance phase. A logistic regression model structure was used for the dropout model.

Model building was done using a non-linear mixed effects approach in the NONMEM software version 7.4.3. The Laplacian estimation method with the likelihood option in the estimation record was used for parameter estimation. Model selection was based on changes in OFV. Model performance was evaluated using visual predictive checks (VPCs) in which 500 replicates of the dataset were simulated and compared to observed data. For the VPCs, additional records at planned visits in the clinical trials were imputed if a patient dropped out before the end of the trial. This allowed for patients to drop out according to model simulations in the VPC. Bootstrapping was performed to evaluate parameter uncertainty. Model diagnostics and SCM were assisted by R version 3.6.0, Perl-speaks-NONMEM (PsN) toolkit version 4.9.0 and Pirana version 2.9.9.

Individual-level longitudinal data from 755 adult patients, placebo/SoC arm, were pooled from five Phase 2/3 clinical trials. The trials were conducted during the years 2006 to 2011. Three of the studies were both induction and maintenance phase studies, and two of the studies were induction phase only. All placebo/SoC arm patients with data in the maintenance phase were also in the placebo/SoC arm during induction phase. Patients had moderate to severe active UC, as evidenced by a MCS of 6 to 12 points and an ENDO subscore of 2 to 3 points at baseline. Across the trials, the RB, SF, and PGA subscores were evaluated every 2-6 weeks, and the ENDO subscore was evaluated at weeks 0, 8, 32/36, and 52. Clinical trial and patient characteristics are summarized in Table 1.

TABLE 1
Baseline characteristic of studies and patients included in modeling analysis

| | | | | | | |
|---|---|---|---|---|---|---|
| Sponsor and Clinical | Abbvie | Abbvie | Abbvie | BMS | Pfizer | |
| Trials.gov number | NCT0038576 | NCT0040869 | NCT00853099 | NCT00410410 | NCT00787202 | Total |

Study Characteristics

| | | | | | | |
|---|---|---|---|---|---|---|
| N (induction maintenance) | 222/—^{a} | 256/143 | 96/57 | 135/20 | 46/—^{a} | 755/220 |
| Induction Phase Duration | 8 | 8 | 8 | 12 | 8 | |
| (weeks) | | | | | | |
| Maintenance Phase Duration | — | 44 | 44 | 40 | — | |
| (weeks) | | | | | | |

Patient Baseline Characteristics

| | | | | | | |
|---|---|---|---|---|---|---|
| Age, years, mean ± SD | 39.7 ± 12.66 | 41.4 ± 13.13 | 41.3 ± 13.56 | 41.2 ± 13.23 | 42.0 ± 13.93 | 40.9 ± 13.11 |
| Mayo Clinical Score, mean ± SD | 8.8 ± 1.60 | 8.9 ± 1.73 | 8.5 ± 1.56 | 8.7 ± 1.56 | 8.3 ± 1.46 | 8.8 ± 1.63 |
| Rectal Bleeding + Stool | 0: 0 (0%) | 0: 1 (0.4%) | 0: 0 (0%) | 0: 0 (0%) | 0: 1 (2.2%) | 0: 2 (0.3%) |
| Frequency Subscore, N (%) | 1: 2 (0.9%) | 1: 4 (1.6%) | 1: 2 (2.1%) | 1: 3 (2.2%) | 1: 0 (0%) | 1: 11 (1.5%) |
| | 2: 21 (9.5%) | 2: 22 (8.6%) | 2: 11 (11.5%) | 2: 16 (11.9%) | 2: 6 (13.0%) | 2: 76 (10.1%) |
| | 3: 47 (21.2%) | 3: 41 (16.0%) | 3: 16 (16.7%) | 3: 31 (23.0%) | 3: 14 (30.4%) | 3: 149 (19.7%) |
| | 4: 67 (30.2%) | 4: 65 (25.4%) | 4: 33 (34.4%) | 4: 36 (26.7%) | 4: 12 (26.1%) | 4: 213 (28.2%) |
| | 5: 59 (26.6%) | 5: 83 (32.4%) | 5: 29 (30.2%) | 5: 37 (27.4%) | 5: 11 (23.9%) | 5: 219 (29.0%) |
| | 6: 26 (11.7%) | 6: 40 (15.6%) | 6: 5 (5.2%) | 6: 11 (8.1%) | 6: 2 (4.3%) | 6: 84 (11.1%) |
| | Missing: 0 (0%) | Missing: 0 (0%) | Missing: 0 (0%) | Missing: 1 (0.7%) | Missing: 0 (0%) | Missing: 1 (0.1%) |
| Endoscopy Subscore, N (%) | 0: 0 (0%) | 0: 0 (0%) | 0: 0 (0%) | 0: 0 (0%) | 0: 0 (0%) | 0: 0 (0%) |
| | 1: 1 (4.5%) | 1: 0 (0%) | 1: 0 (0%) | 1: 0 (0%) | 1: 0 (0%) | 1: 1 (0.1%) |
| | 2: 112 (50.5%) | 2: 138 (53.9%) | 2: 55 (57.3%) | 2: 55 (40.7%) | 2: 24 (52.2%) | 2: 384 (50.9%) |
| | 3: 109 (49.1%) | 3: 118 (46.1%) | 3: 41 (42.7%) | 3: 79 (58.5%) | 3: 22 (47.8%) | 3: 369 (48.9%) |
| | Missing: 0 (0%) | Missing: 0 (0%) | Missing: 0 (0%) | Missing: 1 (0.7%) | Missing: 0 (0%) | Missing: 1 (0.1%) |
| PGA subscore, N (%) | 0: 0 (0%) | 0: 1 (0.4%) | 0: 0 (0%) | 0: 0 (0%) | 0: 0 (0%) | 0: 1 (0.1%) |
| | 1: 8 (3.6%) | 1: 16 (6.3%) | 1: 4 (4.2%) | 1: 5 (3.7%) | 1: 1 (2.2%) | 1: 34 (4.5%) |
| | 2: 155 (69.8%) | 2: 161 (62.9%) | 2: 73 (76.0%) | 2: 94 (69.6%) | 2: 36 (78.3%) | 2: 519 (68.7%) |
| | 3: 59 (26.6%) | 3: 78 (30.5%) | 3: 19 (19.8%) | 3: 36 (26.7%) | 3: 9 (19.6%) | 3: 201 (26.6%) |
| | Missing: 0 (0%) | Missing: 0 (0%) | Missing: 0 (0%) | Missing: 0 (0%) | Missing: 0 (0%) | Missing: 0 (0%) |
| Prior anti-TNF therapy, N (%) | 0 (0%) | 102 (40%) | 0 (0%) | 27 (20%) | 12 (26%) | 141 (19%) |
| Baseline Steroid Use^{b}, (%) | 67.60% | 56.90% | 60.40% | 44.30% | 27% | |
| Concomitant Medications | AS | AS | AS | AS | AS | |
| | AZ/6-MP | AZ/6-MP | AZ/6-MP | AZ/6-MP | Oral steroid | |
| | Oral steroid | Oral steroid | Oral steroid | Oral steroid | | |

^{a}NCT00385736 and NCT00787202 only included an induction phase
^{b}Value obtained from summary provided in publications for each clinical trial
N: number of patients,
SD: standard deviation,
AS: Aminosalicylates,
AZ: Azathioprine,
6-MP: 6-mercaptopurine

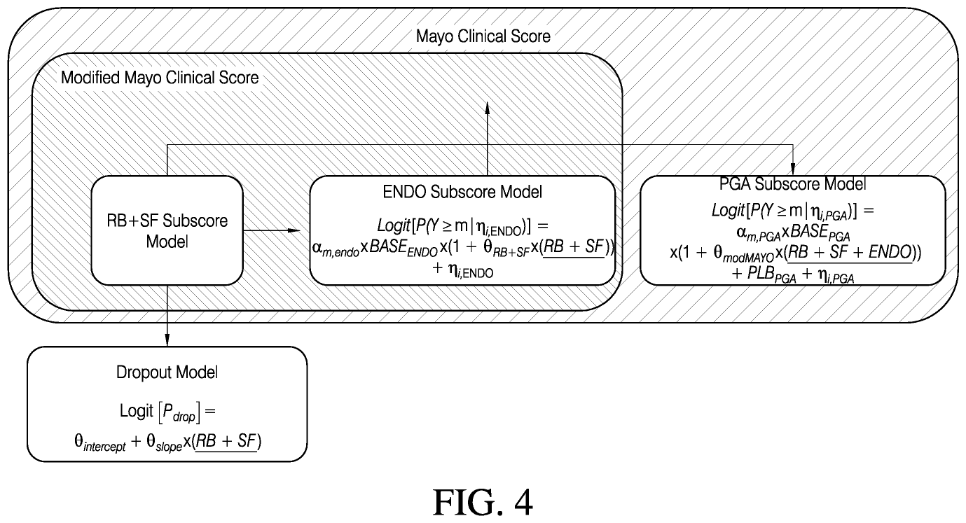

The complete model consisted of three subscore models and one dropout model that were linked together using model-predicted subscores. FIG. 4 shows a schematic of an exemplary UC model structure. The predicted RB+SF subscore informed predictions for the ENDO and PGA subscore models, and the dropout model. The predicted ENDO subscore was combined with the predicted RB+SF subscore to give the modified MCS, which informed the PGA subscore model. General equations representing the linkages between models are shown in FIG. 4.

The MCS subscores were modeled using a PO model. The RB and SF subscores were summed and modeled as one endpoint (RB+SF). A linear placebo effect (PLB=−SLOPE_{PLB}*TIME) was included in the RB+SF subscore model. A linear model was selected as it provided a good description of the data. Estimating separate slopes for induction and maintenance phases as a piecewise linear function resulted in a significant change in the objective function value (OFV) with ΔOFV=−81.1, and this was incorporated in the final model. An exploration of the dataset using the Spearman rank correlation test revealed the RB+SF subscore and ENDO subscore (ρ=0.65), and the modified MCS and PGA subscore were well correlated (ρ=0.80). The model-predicted RB+SF subscore from the same or most recent study visit was therefore included as a time-varying covariate in the ENDO subscore model. This resulted in a significant change in OFV (ΔOFV=−309.4). The ENDO subscore was modeled as a function of the baseline ENDO score and the model-predicted RB+SF subscore. Any time-varying placebo response was hence included indirectly based on the time-varying RB+SF subscore. Parameters for an independent placebo effect on the ENDO subscore could not be estimated. The development of a subscore model was also explored for the PGA subscore. The model-predicted modified MCS, derived by adding predictions from the RB+SF and ENDO subscore models, was included as a time-varying covariate in the PGA subscore model. Inclusion of the modified MCS as a time-varying covariate resulted in a significant change in the OFV (ΔOFV=−609.7). The PGA subscore model included a linear placebo effect, in addition to the placebo effect included in the RB+SF subscore model. Without this additional placebo effect parameter, the model significantly overpredicted the PGA subscore over time. IIV was estimated for the intercept parameter (αₘ) of each of the subscore models. The dataset did not support the estimation of IIV on the placebo slope parameter. Baseline values of the subscores were included as covariates in the corresponding subscore model. In the SCM, the effect of prior exposure to TNF-α antagonists on the RB+SF subscore model was the only covariate retained. None of the tested covariates were identified as significant on the ENDO subscore model. Patients with prior exposure to TNF-α antagonists had higher post-baseline RB+SF subscores than patients who were naïve to TNF-α.

The dropout of patients was estimated at the end of the induction phase, as well as during the maintenance phase. Logistic regression with an intercept parameter, and a slope parameter for the effect of the most recent RB+SF subscore on the probability of dropping out was used, with separate parameter estimates for dropout at the end of induction and during maintenance. With this dropout model, dropout did not occur prior to the end of induction. A positive slope was estimated for both the induction and maintenance phase showing that patients with higher RB+SF subscores had an increased probability of dropping out at the end of induction and during the maintenance phase.

Parameter estimates for the subscore models and dropout model are presented in Table 2. For parameters that can take a positive or negative value, standard error (SE) is presented instead of relative standard error (RSE). Both the SE and RSE in the table were obtained from the NONMVEM covariance step. Non-parametric bootstrapping was performed and for most parameters, resulted in SEs of similar magnitude as those provided by the NONMEM covariance step. SE from bootstrapping were slightly larger for the modified MCS covariate parameter and intercept parameters in the PGA subscore model.

TABLE 2
Model Parameter Estimates

| | | | |
|---|---|---|---|
| Parameter | Estimate | RSE (%) | SE |

RB + SF Subscore PO Model

| | | | |
|---|---|---|---|
| α₁ | 5.34 | | 0.19 |
| DF₂ | −2.34 | 5.3 | |
| DF₃ | −1.32 | 5.3 | |
| DF₄ | −1.37 | 5.1 | |
| DF₅ | −1.66 | 5.2 | |
| DF₆ | −2.27 | 5.9 | |
| SLOPE_{PLB, IND} (1/day) | 0.015 | | 0.0020 |
| SLOPE_{PLB, MAINT} (1/day) | 0.0011 | | 6.4 × 10⁻⁴ |
| BL_RBSF_{α1} | 0.18 | | 0.013 |
| TNF_{α1} | 0.14 | | 0.037 |
| Var(η_{α1}) | 3.47 | 9.4 | |

ENDO Subscore PO Model

| | | | |
|---|---|---|---|
| α_{1, endo} | 0.53 | | 0.17 |
| DF_{2, endo} | −2.84 | 7.3 | |
| DF_{3, endo} | −2.56 | 8.0 | |
| BL_ENDO_{α1, endo} | 0.41 | | 0.073 |
| PDV_RBSF_{α1, endo} | 1.63 | | 0.59 |
| Var(η_{α1, endo}) | 1.01 | 34.1 | |

PGA Subscore PO Model

| | | | |
|---|---|---|---|
| α_{1, PGA} | 0.0093 | | 4.4 × 10⁻⁴ |
| DF_{2, PGA} | −4.56 | 9.0 | |
| DF_{3, PGA} | −4.77 | 10.0 | |
| SLOPE_{PLB, PGA, IND} | 0.016 | | 0.0039 |
| SLOPE_{PLB, PGA, MAINT} | 0.0023 | | 8.6 × 10⁻⁴ |
| BL_PGA_{α1, PGA} | 0.24 | | 0.035 |
| PDV_MMCS_{α1, PGA} | 130 | | 13 |
| Var(η_{α1, PGA}) | 1.21 | 57.2 | |

End of Induction Dropout Logistic Regression Model

| | | |
|---|---|---|
| INTERCEPT_{IND} | −1.94 | 0.25 |
| SLOPE_{IND} | 0.68 | 0.071 |
| INTERCEPT_{MAINT} | −4.65 | 0.31 |
| SLOPE_{MAINT} | 0.84 | 0.072 |

RSE: relative standard error,
SE: standard error,
α1: intercept parameter on the logit scale for score ≥ 1,
DFₖ: parameter for score k such that αₖ = αₖ₋₁ + dfₖ,
SLOPE_{PLB, IND}: slope of the time effect on the subscore during induction phase,
SLOPED_{PLB, MAINT}: slope of the time effect on the subscore during maintenance phase,
BL_RBSF: effect of baseline RB + SF subscore,
TNF: effect of prior anti-TNF treatment,
Var(η): variance of between-subject variability,
BL_ENDO: effect of baseline ENDO subscore,
PDV_RBSF: effect of model-predicted RB + SF subscore,
BL_PGA: effect of baseline PGA subscore,
PDV_MMCS: effect of model-predicted modified MCS,
INTERCEPT_{IND}: intercept of the logistic regression at the end of induction phase,
SLOPE_{IND}: slope of the logistic regression at the end of induction phase,
INTERCEPT_{MAINT}: intercept of the logistic regression during maintenance phase,
SLOPE_{MAINT}: slope of the logistic regression during maintenance phase

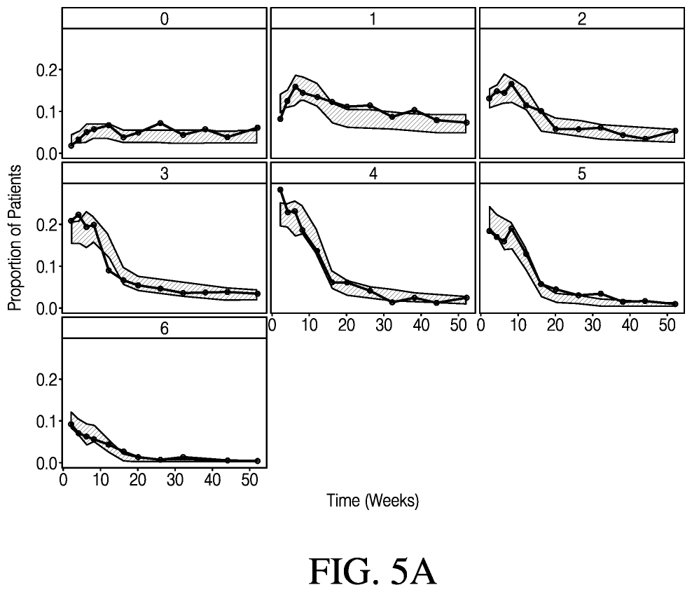

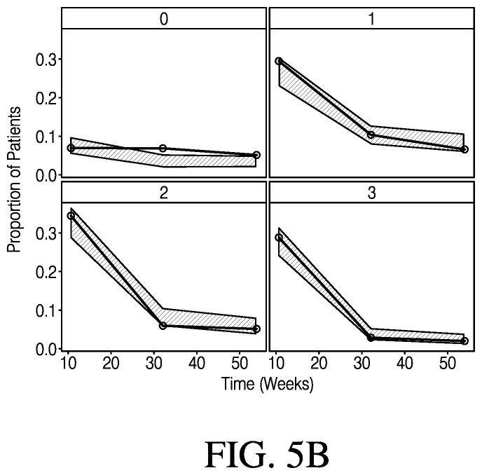

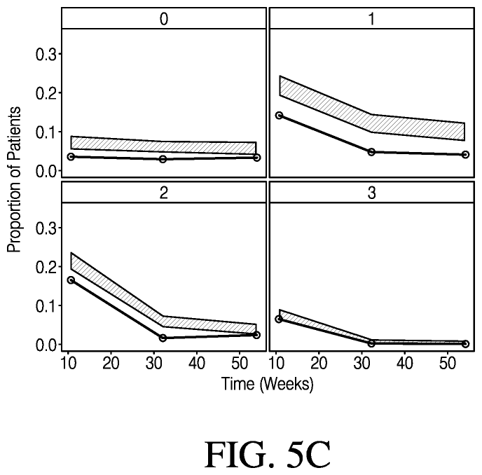

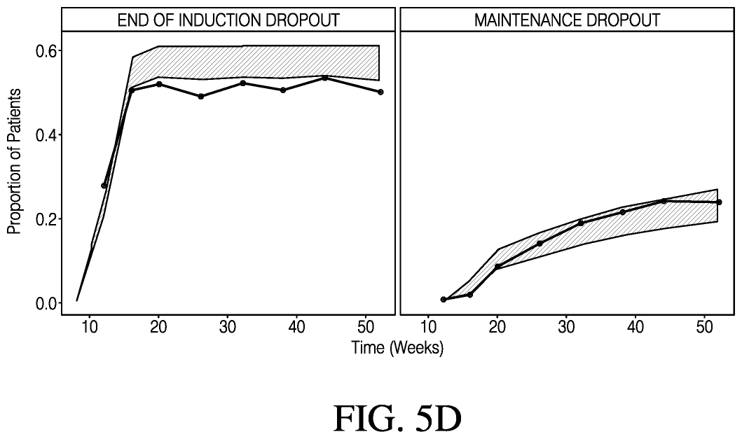

Categorical VPCs were generated for each of the subscore and dropout models. The VPCs show the proportion of patients in each category over time. The proportions for each of the subscore categories accounted for dropout patients and were calculated using the number of patients in each category over the total number of patients possible in the induction/maintenance phase. Because the progression of patients into the maintenance phase in induction-only trials was not possible, the denominator was different for the induction and maintenance phase. FIG. 5A shows categorical VPCs for post-baseline RB+SF subscores. FIG. 5B shows categorical VPCs for post-baseline ENDO subscores. FIG. 5C shows categorical VPCs for post-baseline PGA subscores. FIG. 5D shows categorical VPCs for post-baseline end of induction and maintenance phase dropout. The observed data are represented by the line, and are overlaid on top of shaded areas representing the 95% prediction interval by the model. The y-axis is the proportion of remaining patients in each category to the total patients enrolled in the induction/maintenance phase.

As shown in FIGS. 5A-5D, categorical VPCs of the RB+SF and ENDO subscore models showed an overall good agreement between the predicted 95% confidence interval and observed proportions. For the PGA subscore, a PO model informed by the modified MCS slightly overpredicted the proportion of patients with subscores of 1 and 2. Timepoints started at week 10 for ENDO and PGA due to the first post-baseline ENDO assessment occurring during week 8-12 in the trials. While the dropout model fits the observed data well during the maintenance phase, the model may overpredict the proportion of patients dropping out at the end of induction. The dropout model estimated dropout at a single time point at the end of induction, and the appearance of fluctuations during maintenance phase in the VPC may be a result of data binning and differences between studies in scheduled visits. It should also be noted that the proportions for the ENDO and dropout at a given time point in the VPCs may not add to 1. This may be due to differences in time resolution between the ENDO and dropout assessments. The same is also true for PGA and dropout.

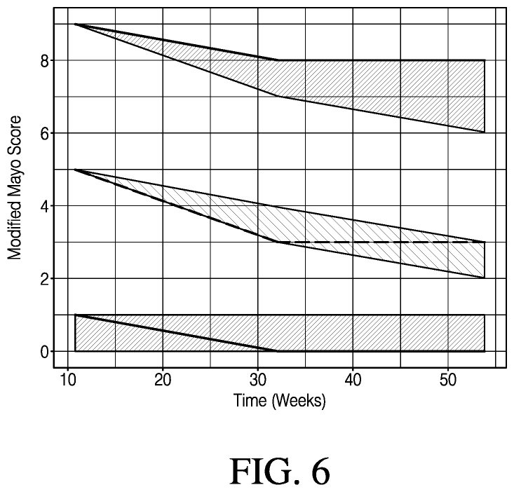

FIG. 6 shows continuous VPC of modified MCS over time in patients remaining in the trial. The median of the observed data is represented as the middle line, and the 2.5th and 97.5th percentiles are represented as the upper and lower lines. Observed data are overlaid with shaded areas representing 95% prediction intervals by the model. Timepoints started at week 10 due to the first post-baseline ENDO assessment occurring during week 8-12 in the trials.

As shown in FIG. 6, the continuous VPC of the modified MCS showed agreement between the predicted 95% confidence interval and observed data. The modified MCS was derived by adding the RB+SF and ENDO estimates at each timepoint. Observed and predicted timepoints demonstrated a decrease in both scores over time. It should be noted that average scores at each timepoint were calculated using only scores from patients remaining in the trial. The dropout of patients (who generally had higher subscores) appears to be a large driver of the decrease in modified MCS over time.

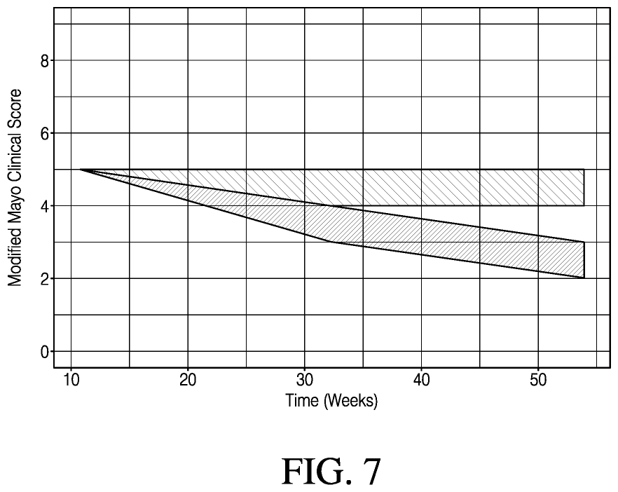

FIG. 7 shows a comparison of model predicted modified MCS with dropout and without dropout. The model with dropout is given by the upper shaded area. The model without dropout is given by the lower shaded area. The comparison of model simulations with dropout and with no dropout of patients demonstrates that the decrease in the modified MCS over time is at least partly driven by the placebo response, and largely driven by the dropout of patients with higher modified MCS from the placebo/SoC arm.

As discussed above, the ordered categorical model was developed to describe the modified MCS and MCS over time in placebo/SoC treated patients with moderate to severe active UC. While the primary objective was to model the modified MCS, modeling of the PGA subscore was explored to allow the model to estimate the MCS over time. RB and SF subscores were combined and modeled as a single endpoint, as the combination of these scores may be a good representation of the symptomatic outcome at each time point. These two subscores are also typically assessed at the same timepoints in clinical trials. The ENDO subscore was estimated as a separate endpoint due to the limited number of ENDO assessments. The model estimates probabilities for the ENDO subscore categories at post-baseline timepoints based on the baseline ENDO subscore and RB and SF subscores from the same visit. The effect of the RB+SF covariate on the ENDO subscore model was statistically significant, and the model was able to produce predictions that agree well with the time course of the observed ENDO data. The change from baseline in ENDO subscore, and therefore the placebo response in the subscore, may be explained by the time-course of RB and SF without the need for an additional parameter for time. The change from baseline in the ENDO subscore, may hence be predicted using only the non-invasive RB and SF subscore evaluations.

The primary objective of this analysis was to model the modified MCS due to a number of considerations associated with the PGA subscore. The PGA subscore may be subjective, it may be unclear what information it provides that is distinct from the other subscores, and regulatory agencies have recently recommended against its use in clinical trials. While the PGA subscore can be evaluated at frequent timepoints in a clinical trial, the PGA subscore may be assigned based on several factors including endoscopic evaluation. Therefore, the PGA subscore model in the current study only estimated this subscore at timepoints in which the ENDO subscore was measured. The additional placebo effect included in the PGA subscore model indicates the PGA subscore may be affected to a greater extent by the placebo effect than the other subscores, and that the change from baseline in PGA subscore may not be estimated from the modified MCS alone. This may be explained by the subjective and variable nature of the PGA subscore. Model misspecification of the PGA subscore may be affected by this subjectivity, and may also be due to the linkages between the subscore and dropout models. Any misspecifications in the RB+SF or ENDO subscore models, or dropout model may affect the PGA subscore model predictions. It should also be noted that the impact of RB+SF and the modified MCS on the subscore and dropout models was based on a continuous slope intercept model. This allowed for a more parsimonious approach involving less model parameters than if the scores were evaluated as categorical covariates on the subscore and dropout models.

In the covariate analysis, patients with prior treatment with TNF-α antagonists had a higher post-baseline RB+SF subscore over time than patients who were naïve to TNF-α antagonists. It should be noted that patients with prior treatment with TNF-α antagonists had similar baseline values of RB and SF subscores as those that were naïve to treatment with TNF-α antagonists. The covariate effect is consistent with the reported difficulty in treating patients with prior treatment with TNF-α antagonists. This should be interpreted with caution, however, as the majority of data for patients with prior treatment with TNF-α antagonists came from a single study in the current dataset. Notably, patient-related factors of age, baseline CRP, baseline serum albumin level, and smoking status, and study-level differences in the proportion of baseline steroid use and in the protocol specifications related to concomitant medications did not have a significant impact on the RB+SF subscore. The lack of age effect may result from the lack of pediatric data, as the current dataset is limited to only adult patients. The effect of steroids and concomitant medications may be difficult to identify due to the lack of patient-level concomitant medication data.

The findings of the dropout model, where patients with higher RB+SF subscores had an increased probability of dropping out at the end of induction, are aligned with the common practices in clinical trials of re-assigning or re-randomizing only patients who responded to the assigned treatment at the beginning of the maintenance phase, and of providing rescue treatment to patients who do not respond. Responder status is typically defined by components of the MCS, where patients with higher scores are non-responders, and those reaching defined lower scores are responders. The dropout model appears to slightly overpredict the dropout of patients at the end of induction, and this is likely due to the variety in mechanisms by which patients are removed from placebo/SoC arms in the clinical trials. For simulations of a single trial with set criteria for dropout, this could potentially be mitigated by mirroring these criteria in the model structure.

Modeling the data sequentially with separate PO models may provide the flexibility to estimate and understand the behavior of individual subscores over time. The RB+SF subscore and PGA subscore models, for example, may be used to predict the partial MCS and interpret early clinical trial data or for interim analyses, when ENDO subscores may not be available. With the complete model, various combinations of MCS subscores such as the modified or partial MCS, may be estimated. The development of the dropout model allowed for the model to capture the complexity in UC clinical trial design, where placebo/SoC arm patients may or may not remain in the placebo/SoC arm during the maintenance phase. Accounting for this aspect of trial design may be needed for accurate estimates of the MCS during the maintenance phase. Modeling the dropout of patients, in addition to the subscores, may also inform sample size estimation in clinical trial design. Alternative model structures (e.g. item response theory, Markov models) were considered for this analysis, but the PO model was selected. The PO model has the simplest structure, and describes the current data well. More complex models were therefore not considered necessary for the current analysis.

The data only included patients who had moderate to severe active UC at baseline. The developed model therefore may not be used to predict outcomes in patients with mild UC. IIV could not be estimated on the linear slope parameter due to insufficient data. The effect of intrinsic and extrinsic factors on disease progression, therefore, could not be evaluated. Because there are no Markov elements to explicitly account for the relationship between serial observations, the model may not be suited for individual-level predictions. The aim of the current model was to generate summary-level predictions of the modified MCS over time, however, and the incorporation of a Markov element was not considered to be necessary. The model may be unable to predict baseline values for the subscores, because baseline values are included as covariates in the subscore models. All of the clinical trials included in the current study were 10 years or older, and trial design and conduct may not be consistent with more recent clinical trials. In particular, the included trials may not have used central endoscopies, and therefore the ENDO data may be more variable than the data from more recent trials. The standard of care has also changed for UC over time, as new therapeutic agents have become available. Placebo/SoC arm patients in more recent clinical trials are thus more likely to have been treated with TNF-α antagonists and other newer therapies than patients from older trials. In addition, because patient-level data for concomitant medications could not feasibly be formatted for modeling, the effect of concomitant medications could only be evaluated using information from the study protocols on the minimum length of treatment prior to study and the medications permitted in the study.

The models described herein may be improved by incorporating additional, more recent clinical trial data to update the model. Considering the additional challenges in pediatric drug development for UC, the availability of pediatric data may also allow for a better understanding of age effects on placebo response. A robust model may generate predictions that can be used as a “virtual placebo arm” for pediatric trials in which a placebo/SoC arm is not feasible to enroll. The model may also have a drug effect parameter incorporated into it, so that it may be applied to data from treatment arm patients. Drug effect may be evaluated categorically, by including separate slopes for the linear effect (Effect=−SLOPE×TIME) for patients in the treatment and placebo arm. If an examination of the concentration-response relationship was of interest, observed or population PK model predicted values may be accounted for in the current model using various approaches such as an effect compartment or indirect response model. The current study pooled data from five clinical trials to develop a longitudinal model describing the modified MCS over time in placebo/SoC arm patients with moderate to severe active UC. By providing insights into the time course of placebo response, and factors that influence the response, the model may support clinical trial design and data interpretation.

### Recitation of Embodiments

Embodiment 1. A method comprising:
receiving a set of time points;
   using a first statistical operation to compute a set of predicted rectal bleeding (RB) scores and a set of predicted stool frequency (SF) scores corresponding to the set of time points; and
   using a second statistical operation to compute a set of predicted mucosal appearance at endoscopy (ENDO) scores corresponding to the set of time points based upon the predicted RB scores and the predicted SF scores.

Embodiment 2. The method of Embodiment 1, further comprising generating a predicted Mayo Clinical Score (MCS) based upon the predicted RB scores, the predicted SF scores, and the predicted ENDO scores.

Embodiment 3. The method of Embodiment 1 or 2, further comprising using a third statistical operation to generate a set of predicted physician's global assessment (PGA) scores corresponding to the set of time points based upon the predicted RB scores, the predicted SF scores, and the predicted ENDO scores.

Embodiment 4. The method of any one or Embodiments 1-3, further comprising using a fourth statistical model to generate a set of predicted dropout likelihood metrics corresponding to the set of time points based upon the predicted RB scores and the predicted SF scores.

Embodiment 5. The method of Embodiment 4, further comprising providing a clinical trial recommendation based upon the set of predicted dropout likelihood metrics.

Embodiment 6. The method of any one of Embodiments 1-5, further comprising generating the first, second, third, or fourth statistical operations based upon training data comprising one or more members selected from the group consisting of: Mayo Clinical Score (MCS) data, modified MCS data, RB score data, SF score data, ENDO score data, and PGA score data.

Embodiment 7. The method of any one of Embodiments 1-6, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

Embodiment 8. The method of any one of Embodiments 1-7, wherein the first, second, third, or fourth statistical operations comprise one or more members selected from the group consisting of: pharmacometric operations, artificial intelligence (AI) operations, proportional odds (PO) operations, and logistic regression operations.

Embodiment 9. A system comprising:
a non-transitory memory; and
one or more processor coupled to the non-transitory memory and configured to read instructions from the non-transitory memory to cause the system to perform operations comprising:
   receiving a set of time points;
   using a first statistical operation to compute a set of predicted rectal bleeding (RB) scores and a set of predicted stool frequency (SF) scores corresponding to the set of time points; and
   using a second statistical operation to compute a set of predicted mucosal appearance at endoscopy (ENDO) scores corresponding to the set of time points based upon the predicted RB scores and the predicted SF scores.

Embodiment 10. The system of Embodiment 9, wherein the operations further comprise generating a predicted Mayo Clinical Score (MCS) based upon the predicted RB scores, the predicted SF scores, and the predicted ENDO scores.

Embodiment 11. The system of Embodiment 9 or 10, wherein the operations further comprise using a third statistical operation to generate a set of predicted physician's global assessment (PGA) scores corresponding to the set of time points based upon the predicted RB scores, the predicted SF scores, and the predicted ENDO scores.

Embodiment 12. The system of any one or Embodiments 9-11, wherein the operations further comprise using a fourth statistical model to generate a set of predicted dropout likelihood metrics corresponding to the set of time points based upon the predicted RB scores and the predicted SF scores.

Embodiment 13. The system of Embodiment 12, wherein the operations further comprise providing a clinical trial recommendation based upon the set of predicted dropout likelihood metrics.

Embodiment 14. The system of any one of Embodiments 9-13, wherein the operations further comprise generating the first, second, third, or fourth statistical operations based upon training data comprising one or more members selected from the group consisting of: Mayo Clinical Score (MCS) data, modified MCS data, RB score data, SF score data, ENDO score data, and PGA score data.

Embodiment 15. The system of any one of Embodiments 9-14, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

Embodiment 16. The system of any one of Embodiments 9-15, wherein the first, second, third, or fourth statistical operations comprise one or more members selected from the group consisting of: pharmacometric operations, artificial intelligence (AI) operations, proportional odds (PO) operations, and logistic regression operations.

Embodiment 17. A non-transitory, machine-readable medium having stored thereon machine-readable instructions executable to cause a system to perform operations comprising:
receiving a set of time points;
using a first statistical operation to compute a set of predicted rectal bleeding (RB) scores and a set of predicted stool frequency (SF) scores corresponding to the set of time points; and
using a second statistical operation to compute a set of predicted mucosal appearance at endoscopy (ENDO) scores corresponding to the set of time points based upon the predicted RB scores and the predicted SF scores.

Embodiment 18. The non-transitory, machine-readable medium of Embodiment 17, wherein the operations further comprise generating a predicted Mayo Clinical Score (MCS) based upon the predicted RB scores, the predicted SF scores, and the predicted ENDO scores.

Embodiment 19. The non-transitory, machine-readable medium of Embodiment 17 or 18, wherein the operations further comprise using a third statistical operation to generate a set of predicted physician's global assessment (PGA) scores corresponding to the set of time points based upon the predicted RB scores, the predicted SF scores, and the predicted ENDO scores.

Embodiment 20. The non-transitory, machine-readable medium of any one or Embodiments 17-19, wherein the operations further comprise using a fourth statistical model to generate a set of predicted dropout likelihood metrics corresponding to the set of time points based upon the predicted RB scores and the predicted SF scores.

Embodiment 21. The non-transitory, machine-readable medium of Embodiment 20, wherein the operations further comprise providing a clinical trial recommendation based upon the set of predicted dropout likelihood metrics.

Embodiment 22. The non-transitory, machine-readable medium of any one of Embodiments 17-21, wherein the operations further comprise generating the first, second, third, or fourth statistical operations based upon training data comprising one or more members selected from the group consisting of: Mayo Clinical Score (MCS) data, modified MCS data, RB score data, SF score data, ENDO score data, and PGA score data.

Embodiment 23. The non-transitory, machine-readable medium of any one of Embodiments 17-22, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

Embodiment 24. The non-transitory, machine-readable medium of any one of Embodiments 17-23, wherein the first, second, third, or fourth statistical operations comprise one or more members selected from the group consisting of: pharmacometric operations, artificial intelligence (AI) operations, proportional odds (PO) operations, and logistic regression operations.

What is claimed is:

1) A method comprising:
receiving a set of time points;
using a first statistical operation to compute a set of predicted rectal bleeding (RB) scores and a set of predicted stool frequency (SF) scores corresponding to the set of time points; and
using a second statistical operation to compute a set of predicted mucosal appearance at endoscopy (ENDO) scores corresponding to the set of time points based upon the predicted RB scores and the predicted SF scores.

2) The method of claim 1, further comprising generating a predicted Mayo Clinical Score (MCS) based upon the predicted RB scores, the predicted SF scores, and the predicted ENDO scores.

3) The method of claim 1, further comprising using a third statistical operation to generate a set of predicted physician's global assessment (PGA) scores corresponding to the set of time points based upon the predicted RB scores, the predicted SF scores, and the predicted ENDO scores.

4) The method of claim 1, further comprising using a fourth statistical model to generate a set of predicted dropout likelihood metrics corresponding to the set of time points based upon the predicted RB scores and the predicted SF scores.

5) The method of claim 4, further comprising providing a clinical trial recommendation based upon the set of predicted dropout likelihood metrics.

6) The method of claim 1, further comprising generating the first, second, third, or fourth statistical operations based upon training data comprising one or more members selected from the group consisting of: Mayo Clinical Score (MCS) data, modified MCS data, RB score data, SF score data, ENDO score data, and PGA score data.

7) The method of claim 1, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

8) The method of claim 1, wherein the first, second, third, or fourth statistical operations comprise one or more members selected from the group consisting of: pharmacometric operations, artificial intelligence (AI) operations, proportional odds (PO) operations, and logistic regression operations.

9) A system comprising:
a non-transitory memory; and
one or more processor coupled to the non-transitory memory and configured to read instructions from the non-transitory memory to cause the system to perform operations comprising:
receiving a set of time points;
using a first statistical operation to compute a set of predicted rectal bleeding (RB) scores and a set of predicted stool frequency (SF) scores corresponding to the set of time points; and
using a second statistical operation to compute a set of predicted mucosal appearance at endoscopy (ENDO) scores corresponding to the set of time points based upon the predicted RB scores and the predicted SF scores.

10) The system of claim 9, wherein the operations further comprise generating a predicted Mayo Clinical Score (MCS) based upon the predicted RB scores, the predicted SF scores, and the predicted ENDO scores.

11) The system of claim 9, wherein the operations further comprise using a third statistical operation to generate a set of predicted physician's global assessment (PGA) scores corresponding to the set of time points based upon the predicted RB scores, the predicted SF scores, and the predicted ENDO scores.

12) The system of claim 9, wherein the operations further comprise using a fourth statistical model to generate a set of predicted dropout likelihood metrics corresponding to the set of time points based upon the predicted RB scores and the predicted SF scores.

13) The system of claim 12, wherein the operations further comprise providing a clinical trial recommendation based upon the set of predicted dropout likelihood metrics.

14) The system of claim 9, wherein the operations further comprise generating the first, second, third, or fourth statistical operations based upon training data comprising one or more members selected from the group consisting of: Mayo Clinical Score (MCS) data, modified MCS data, RB score data, SF score data, ENDO score data, and PGA score data.

15) The system of claim 9, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

16) The system of claim 9, wherein the first, second, third, or fourth statistical operations comprise one or more members selected from the group consisting of: pharmacometric operations, artificial intelligence (AI) operations, proportional odds (PO) operations, and logistic regression operations.

17) A non-transitory, machine-readable medium having stored thereon machine-readable instructions executable to cause a system to perform operations comprising:
receiving a set of time points;
using a first statistical operation to compute a set of predicted rectal bleeding (RB) scores and a set of predicted stool frequency (SF) scores corresponding to the set of time points; and
using a second statistical operation to compute a set of predicted mucosal appearance at endoscopy (ENDO) scores corresponding to the set of time points based upon the predicted RB scores and the predicted SF scores.

18) The non-transitory, machine-readable medium of claim 17, wherein the operations further comprise generating a predicted Mayo Clinical Score (MCS) based upon the predicted RB scores, the predicted SF scores, and the predicted ENDO scores.

19) The non-transitory, machine-readable medium of claim 17, wherein the operations further comprise using a third statistical operation to generate a set of predicted physician's global assessment (PGA) scores corresponding to the set of time points based upon the predicted RB scores, the predicted SF scores, and the predicted ENDO scores.

20) The non-transitory, machine-readable medium of claim 17, wherein the operations further comprise using a fourth statistical model to generate a set of predicted dropout likelihood metrics corresponding to the set of time points based upon the predicted RB scores and the predicted SF scores.

21) The non-transitory, machine-readable medium of claim 20, wherein the operations further comprise providing a clinical trial recommendation based upon the set of predicted dropout likelihood metrics.

22) The non-transitory, machine-readable medium of claim 17, wherein the operations further comprise generating the first, second, third, or fourth statistical operations based upon training data comprising one or more members selected from the group consisting of: Mayo Clinical Score (MCS) data, modified MCS data, RB score data, SF score data, ENDO score data, and PGA score data.

23) The non-transitory, machine-readable medium of claim 17, wherein at least one of the set of predicted RB scores, the set of predicted SF scores, the set of predicted ENDO scores, and the set of predicted PGA scores comprises a set of predicted distributions of RB scores, a set of predicted distributions of SF scores, a set of predicted distributions of ENDO scores, or a set of predicted distributions of PGA scores, respectively.

24) The non-transitory, machine-readable medium of claim 17, wherein the first, second, third, or fourth statistical operations comprise one or more members selected from the group consisting of: pharmacometric operations, artificial intelligence (AI) operations, proportional odds (PO) operations, and logistic regression operations.
